(19) United States
(12) Patent Application Publication
(10) Pub. No.: US 2014/0227366 A1
(43) Pub. Date: Aug. 14, 2014

(54) CARRIER MATERIAL FOR IMPROVING THE PERSISTANCE OF BIOCIDES

(71) Applicants: Oliver Zindel, Dreieich (DE); Karla Kaminski, Essen (DE); Frank Iding, London (GB); Volker Windhoevel, Borken (DE)

(72) Inventors: Oliver Zindel, Dreieich (DE); Karla Kaminski, Essen (DE); Frank Iding, London (GB); Volker Windhoevel, Borken (DE)

(21) Appl. No.: 14/117,459

(22) PCT Filed: May 11, 2012
(86) PCT No.: PCT/EP12/58755

§ 371 Date: Mar. 21, 2014

(30) Foreign Application Priority Data
May 13, 2011 (EP) 11166081.7

(51) Int. Cl. A61K 9/70; A61K 47/36; A61K 31/5395

(52) U.S. Cl. CPC A61K 9/7007; A61K 31/5395; A61K 47/36
USPC 424/604; 514/229.2; 514/777

(57) ABSTRACT

The present invention relates to a biocide improvement system wherein the biocide is reversibly bound into a matrix of an organic polymer and inorganic solid particles and thus is slowly released in the soil.

### CROSS-REFERENCES TO RELATED APPLICATIONS

This application is a National Stage filing of International Application PCT/EP 2012/058755, filed May 11, 2012, claiming priority to EP Application No. 11166081.7 filed May 13, 2011, entitled “CARRIER MATERIAL FOR IMPROVING THE PERSISTENCE OF BIOCIDES.” The subject application claims priority to PCT/EP 2012/058755, and to EP Application No. EP 11166081.7 and incorporates all by reference herein, in their entirety.

### BACKGROUND OF THE INVENTION

The present application relates to the field of aids and improvement systems for biocides in soils.

When using biocides in soils, the problem occurs that because of the properties of the biocides (such as solubility in water, etc.), which are often necessary for the efficacy of the biocides, the efficacy of biocides and the toxicity for the surroundings is increased in that biocides are washed out too rapidly and can get into the groundwater or other undesirable soil areas.

Furthermore, there is the problem that biocides are used or can only be used if plants have already grown in the areas to be treated, and that subsequent introduction of biocides into the soil, for example, is no longer possible.

### DETAILED DESCRIPTION OF THE INVENTION

The task therefore arises of creating a biocide improvement system with which the availability of the biocides can be maintained or worsened only insignificantly, on the one hand, but a retarding effect can be set, on the other hand.

This task is accomplished by a biocide improvement system according to claim 1 of the present invention. According to this claim, a biocide improvement system for use in soils is proposed, comprising
a water-swelling matrix material on the basis of at least one organic polymer, which comprises carbohydrate-based structural units, particularly carbohydrate-based functional groups,
wherein inorganic solid particles are added to the matrix material,
and comprising at least one biocide reversibly bound to the matrix material and/or to the inorganic solid particles.

Surprisingly, it has been shown that in most uses of such a biocide improvement system, one or more of the following advantages can be achieved:
The release of the biocide is delayed over time, and “washing out” also takes place only to a lesser extent.
The amount of biocide required can be reduced.
Reversible binding of the biocide can take place in simple manner, in that the biocide is added before or during polymerization of the matrix material.
The degree of efficacy of the biocide is improved.
Application of the biocide takes place directly into the soil.
As a result of the introduction of the biocide bound to the carrier material, environmental contamination (transfer by means of wind or rain, cross-contamination of non-treated areas and/or animals) is reduced.
Because of the presence in the soil, more uniform application and/or absorption takes place, thereby improving the efficiency of the biocide.
Because of the improvement system according to the invention, it is possible that biocides are only batched up in the correct concentration “on site”: The risk that proceeds from transport (of hazardous materials) is reduced, since the biocide is present and is transported at the low application concentration. In the case of sprays, there is therefore less risk of excess residual spray material from overly large batches, as a result of the system according to the invention; cleaning of used equipment is eliminated or simplified.
Even biocides that are actually incompatible with one another can be combined with one another in the biocide improvement system, and can be used in time-saving and effort-saving manner.

The term “biocide” in the sense of the present invention should be understood in the broadest sense and particularly comprises all biocides, particularly those with a low molecular weight, which can be used in soils. Preferred biocides are fungicides, pesticides, herbicides and/or insecticides. It should be pointed out that the present invention is not restricted to a class of biocides, but rather, instead, can be used for almost all substance classes, because it has been shown that the effect of the improvement system is more universal than specific.

The term “reversibly bound to the matrix material and/or to the inorganic solid particles” should also be understood in the broadest sense and, in this connection, comprises not only a physical bond, such as simple embedding into the inorganic solid particles (if necessary, also by way of solution in water that is embedded in the solid particles), reversible adsorption or absorption, but also a chemical bond, either by way of salt formation, electrostatic or hydrogen bridge bonds, or by way of (reversible) covalent bonds or metal complexes.

In this connection, the term “reversible” should particularly be understood to mean that the biocide is completely, i.e. by more than 98%, preferably 99%, released in the soil over an extended period of time (time periods of 1-5 years). This can also take place earlier, depending on the application.

Preferably, the biocide is selected from the group containing aliphatic nitrogen compounds, antibiotic fungicides, macrocyclic lactones, amides, anilides, particularly acylamino acids, inorganic herbicides, chloroacetanilides, phenoxynicotinanalides, sulfonanilide, anilino-pyrimidines, aryloxyphenoxypropionates, aryl-phenyl-ketones, azoles, benzofuranylalkylsulfonate, benzothiazole, bipyridyls, carbamates, carbanilates, growth regulators, chitin synthesis inhibitors, chlorinated hydrocarbons, cyclohexanediones, cyclohexene oximes, diazoles, dichlorophenyl dicarboximides, dinitroanilines, dinitrophenol, diphenyl ethers, dithiolanes, halogenated aliphates, urea derivatives, hydrazides, imidazolinones, Juvenile Hormone, synthetic Juvenile Hormones, coumarins, morpholines, neonicotinoids, nitriles, nitrophenyl ethers, organophosphates, organothiophosphates, oxadiazoles, oxazolidinone derivatives, phenoxy herbicides, phenyl pyrazolines, pheromones, phosphoric acid esters, phthalimides, Precocene, pyrazoles, pyrethroids, pyridazines, pyridazinones, pyridines, pyromidines, pyrroles, quaternary ammonium salts, quinolines, quinoxalines, quinones, strobilurins, sulfite esters, sulfonyl ureas, synthetic auxins, tetrazines, tetronic acid, thiadiazines, thiophene, thiazolidines, triazines, triazinones, triazoles, triazolones, or mixtures thereof.

In this connection, it has been shown that the different substance groups are generally bound to the matrix material and/or the inorganic solid particles as follows, in the case of most applications:

| Substance Group | Type of Main Bond |
|---|---|
| Amides | salt formation |
| Anilides | salt formation, esterification |
| Anilino-pyrimidines | condensation |
| Aryl-phenyl-ketones | grafting |
| Aryloxyphenoxypropionates | condensation |
| Azoles | salt formation, ring digestion |
| Bipyridyls | salt formation |
| Carbamates | salt formation/covalent bond/ |
| | condensation |
| Carbanilates | esterification |
| Chitin synthesis inhibitors | salt formation/covalent bond/ |
| | condensation |
| Chloroacetanilides | salt formation |
| Cyclohexanediones | esterification |
| Cyclohexene oximes | condensation |
| Dichlorophenyl dicarboximides | esterification |
| Dinitroanilines | condensation |
| Urea derivatives | condensation |
| Coumarins | grafting |
| Morpholines | grafting |
| Neonicotinoids | salt formation/grafting |
| Nitriles | esterification |
| Nitrophenyl ether | esterification |
| Organophosphates | esterification |
| Organothiophosphates | esterification |
| Oxazolidinone derivatives | grafting, cross-linking |
| Phenoxy nicotine anilides | salt formation |
| Phenyl pyrazolines | esterification |
| Pheromones | grafting |
| Phosphoric acid esters | grafting, esterification |
| Phthalimides | salt formation |
| Pyrazoles | salt |
| Pyrethroids | grafting |
| Pyridazinones | salt formation |
| Pyridines | salt formation |
| Pyrimidines | salt formation/covalent bond/ |
| | condensation |
| Pyrroles | condensation |
| Quinones | grafting |
| Strobilurins | grafting |
| Sulfonanilide | salt formation |
| Sulfonyl ureas | condensation |
| Synthetic auxins | condensation |
| Tetronic acid | tetronic acid esterification |
| Thiadiazines | salt formation/covalent bond/ |
| | condensation |
| Thiazolidines | salt |
| Triazines | grafting |
| Triazinones | grafting |
| Triazoles | salt |
| Acylamino acids | condensation/hydrolysis |
| Inorganic herbicides | salt formation |
| Benzofuranylalkylsulfonate | grafting |
| Benzothiazole | grafting |
| Chlorinated hydrocarbons | grafting |
| Diazoles | grafting |
| Dinitrophenol | esterification |
| Diphenyl ether | esterification |
| Halogenated aliphates | esterification |
| Imidazolinones | esterification |
| Juvenile hormones | grafting |
| Macrocyclic lactone-abamectins | esterification |
| Nitriles | condensation/esterification |
| Oxadiazoles | grafting |
| Precocene | grafting |
| Phenoxy herbicides | grafting |
| Pyridazines | salt formation |
| Quaternary ammonium salts | grafting |
| Quinoxalines | salt formation |
| Sulfite esters | salt formation |
| Synthetic Juvenile Hormones | grafting |
| Dithiolanes | grafting |
| Hydrazides | grafting |
| Quinolines | grafting |
| Thiophene | grafting |
| Tetrazines | grafting |
| Triazolones | grafting |

According to a preferred embodiment of the invention, the concentration of biocide amounts to from ≧0.0001% to ≦10% (weight of biocide/weight of matrix material+solid particles), particularly ≧0.001% to ≦5% (weight of biocide/weight of matrix material+solid particles), more preferably ≧0.01% to ≦1% (weight of biocide/weight of matrix material+solid particles).

According to a preferred embodiment of the invention, the biocide is selected from the group including (E)7-(Z)9-dodecadienyl acetate, abamectin, aclonifen, acrolein, alpha-cypermethrin, aluminum phosphide, amidosulfuron, azoxystrobin, bendiocarb, bentazon, benzofuranyl methylcarbamate, beta-cyfluthrin, Bitrex, boroxide, boric acid, boscalid, bromadiolone, bromoxynil, bromoxynil octanoate, captan, carbaryl, carfentrazone-ethyl, chalcogran, chloralose, chloridazon, chloromequat chloride, chlorothalonil, chlorophacinone, clodinafop propargyl, clomazone, cloquintocet-mexyl, clothianidin, codlemone, coumatetralyl, cycloxydim, cyproconazole, cyprodinil, dazomet, diquat, diquat bromide, deltamethrin, desmedipham, dicamba, dichofluanid, dichloroprop-P, dichlorvos, difenacoum, difenoconazole, difethialone, diflufenican, dimethachlor, dimethenamid-P, dimethoate, dimethomorph, dimethyl carbamate dimoxystrobin, disodium octaborate tetrahydrate, disodium tetraborate, dithianon, epoxiconazole, esfenvalerate, etofenprox ethephon, ethofumesate, ethylenediamine hydroxyphenyl acetic acid, etofenprox, fenhexamid, fenoxycarb, fenpropidin, fenpropimorph, flocoumafen, florasulam, fluazifop-P-butyl, fluazinam, fludioxonil, flufenacet, fluopicolide, fluoxastrobin, fluquinconazole, fluoroxypyr, flurtamone, folpet, foramsulfuron, fosetyl-A1, fosthiazate, fuberidazole, glufosinate ammonium, glyphosate, hexythiazox, imazalil, imidacloprid, indoxacarb, 3-iodo-2-propynylbutylcarbamate, iodosulfuron methyl sodium, ioxynil, iprodione, iprovalicarb, isoproturon, isoxadifen ethyl, kresoxim methyl, lambda cyhalothrin, magnesium phosphide, mancozeb, mandipropamid, mefenpyr-diethyl, mepiquat chloride, mesosulfuron methyl, mesotrione, metafluizone, metalaxyl-M, metaldehyde, metazachlor, metconazole, methiocarb, methofluthrin, methoxyfenozide, metiram, metosulam, metrafenone, metribuzin, napropamide, N,N-diethyl-m-toluamide, penconazole, pencycuron, penoxsulam, pethoxamid, phenmedipham, picolinafen, pinoxaden, piperonyl butoxide, pirimicarb, pirimiphos methyl, prochloraz, prochloraz copper chloride, prohexadione calcium, propamocarb HCl, propamocarb HCl, propiconazole, propoxycarbazone, propylene glycol, proquinazid, prosulfocarb, prosulfuron, prothioconazole, pymetrozine, pyraclostrobin, pyrethrin, pyrimethanil, quinmerac, quizalofop-P, glyphosate, S-cis-verbenol, S-ipsdienol, S-metolachlor, spinosad, spirodiclofen, spiroxamine, sulfuryl fluoride, sulcotrione, tebuconazole, tebufenpyrad, tefluthrin, tembotrione, tepraloxydim, terbuthylazine, thiabendazole, thiacloprid, thiamethoxam, thiophanate methyl, tolylfluanid, topramezone, triasulfuron, trifloxystrobin, triflumuron, trinexapac-ethyl, triticonazole, tritosulfuron, warfarin, warfarin sodium, A-9 dodecenyl acetate, zinc phosphide, or mixtures of these substances.

According to a preferred embodiment of the invention, the organic polymer is configured to be cross-linked, particularly structurally cross-linked. This has proven to be particularly advantageous because in this way, the possibilities of controlled release of substances bound in the polymer can often be increased.

According to a preferred embodiment of the invention, cross-linking is brought about by means of difunctional cross-linking agents, particularly by means of diols.

According to a preferred embodiment of the invention, the carbohydrate-based structural units are bound to the organic polymer, particularly chemically bound, preferentially by means of grafting or condensation, particularly esterification.

According to a preferred embodiment of the invention, the organic polymer possesses a sponge-like and/or porous structure, particularly one having cavities. Preferentially, in this connection, the inorganic solid particles are embedded into the organic polymer and/or the inorganic solid particles are bound to the organic polymer. This embodiment has proven itself in practice, particularly for the cases in which no or only a weak chemical bond is present between the matrix material or the inorganic particles, respectively, and the biocide, but rather, above all, there is a physical bond. By means of the increase in size of the surface, more surface area is available for “agglomeration.”

According to a preferred embodiment of the invention, the organic polymer is biocompatible, particularly biodegradable, preferentially under the effect of microorganisms.

According to a preferred embodiment of the invention, the organic polymer is configured to be hydrophilic. This increases the bond of polar and/or water-soluble biocides to the polymer, in many embodiments.

According to a preferred embodiment of the invention, the organic polymer is configured on the basis of at least one super-absorbing polymer (SAP).

According to a preferred embodiment of the invention, the organic polymer contains or comprises a homopolymer and/or copolymer of at least one ethylene-unsaturated organic compound, particularly of acrylic acid, methacrylic acid or their derivatives.

According to a preferred embodiment of the invention, the organic polymer contains or comprises a polymer that contains carboxyl groups. This has proven to be advantageous, because in this way, bonding of many biocides to the polymer can be increased (either by means of salt formation or by means of esterification/condensation).

According to a preferred embodiment of the invention, the organic polymer contains or comprises a polymer that is derived from at least one unsaturated carboxylic acid, particularly an aliphatic, aromatic-aliphatic or aromatic unsaturated carboxylic acid, preferably an aliphatic unsaturated carboxylic acid, particularly preferably from the group of acrylic acid, methacrylic acid as well as mixtures and esters thereof, particularly preferably acrylic acid and its esters.

According to a preferred embodiment of the invention, the organic polymer contains or comprises a polyacrylate or polymethacrylate, preferably a cross-linked, particularly structurally cross-linked polyacrylate. It has been shown that in many embodiments, transverse cross-linking has positive effects on control of release of the biocide in the soil.

According to a preferred embodiment of the invention, the carbohydrate-based structural units are covalently bound to the organic polymer, preferentially condensed, particularly by means of esterification.

In this connection, preferably ≧0.1%, preferentially ≧1%, preferably ≧5% and/or particularly ≦80%, preferentially ≦50%, particularly preferably ≦20% of the carboxylic acid functions of the organic polymer are esterified with carbohydrate-based structural units and/or particularly wherein ≧0.1% to ≦80%, preferentially ≧1% to ≦50%, preferably ≧5% to ≦20% of the carboxylic acid functions of the organic polymer are esterified with carbohydrate-based structural units.

According to a preferred embodiment of the invention, the organic polymer contains or comprises a particularly cross-linked, preferentially transversely cross-linked polyacrylate or polymethacrylate, wherein ≧0.1%, preferentially ≧1%, preferably ≧2% and/or particularly ≦80%, preferentially ≦50%, particularly preferably ≦20% of the carboxylic acid functions of the organic polymer are esterified with carbohydrate-based structural units and/or wherein ≧0.1% to ≦80%, preferentially ≧1% to ≦50%, preferably ≧2% to ≦20% of the carboxylic acid functions of the organic polymer are esterified with carbohydrate-based structural units.

According to a preferred embodiment of the invention, the organic polymer contains carbohydrate-based structural units in a weight ratio of organic polymer/carbohydrate-based structural units ≧1:1, particularly ≧2:1, preferentially ≧2.5:1, particularly preferably ≧3:1, very particularly preferably ≧4:1, and/or that the organic polymer contains carbohydrate-based structural units in a weight ratio of organic polymer/carbohydrate-based structural units in the range from 1:1 to 500:1, particularly 2:1 to 200:1, preferentially 3:1 to 100:1, particularly preferably 4:1 to 10:1.

According to a preferred embodiment of the invention, the organic polymer contains a particularly cross-linked, preferentially transversely cross-linked polyacrylate or polymethacrylate, wherein the organic polymer contains carbohydrate-based structural units in a weight ratio of organic polymer/carbohydrate-based structural units of ≧1:1, particularly ≧1%, preferably ≧2:1, preferentially ≧2.5:1, particularly preferably ≧3:1, very particularly preferably ≧4:1 and/or wherein the organic polymer contains carbohydrate-based structural units in a weight ratio of organic polymer/carbohydrate-based structural units in the range of 1:1 to 500:1, particularly 2:1 to 200:1, preferentially 3:1 to 100:1, particularly preferably 4:1 to 10:1.

According to a preferred embodiment of the invention, the biocide improvement system contains carbohydrate-based structural units, with reference to the biocide improvement system, in amounts of ≧0.01 to ≦40 wt.-%, particularly ≧0.2 to ≦30 wt.-%, preferentially ≧0.4 to ≦25 wt.-%, particularly preferably ≧0.51 to ≦10 wt.-%.

According to a preferred embodiment of the invention, carbohydrate-based structural units are configured the same or differently. In this connection, it is preferred that in the case of structural units that are different from one another, at least two, preferentially at least three structural units that are different from one another are present.

According to a preferred embodiment of the invention, the carbohydrate-based structural units are configured on the basis of saccharide bonds, particularly from the group of monosaccharides, disaccharides, oligosaccharides, and polysaccharides, and mixtures thereof.

According to a preferred embodiment of the invention, the carbohydrate-based structural units are configured on the basis of organic compounds having a carbonyl group that forms a hemiacetal, and, at the same time, multiple hydroxy groups in the molecule, particularly polyhydroxy aldehydes (aldoses) and polyhydroxy ketones (ketoses), as well as compounds derived from these, as well as their oligocondensates and polycondensates.

According to a preferred embodiment of the invention, the carbohydrate-based structural units are configured on the basis of compounds that are selected from the group of glucose; saccharose; cellulose and cellulose derivatives, particularly cellulose ethers and esters; starch and starch derivatives, particularly starch ethers; molasses as well as its mixtures.

According to a preferred embodiment of the invention, the carbohydrate-based structural units are configured on the basis of glycans, preferably homoglycans.

According to the invention, the biocide improvement system comprises inorganic solid particles. In this connection, inorganic solid particles that are the same or different from one another can be present in the biocide improvement system. In the case of inorganic solid particles that are different from one another, preferably at least two, preferentially at least three inorganic solid particles that are different from one another are present.

According to a preferred embodiment of the invention, the inorganic solid is selected from among mineral rocks, particularly rock meals and/or a finely ground form.

Preferably, the inorganic solid is selected from the group of basalt, bentonite, pumice, calcite, carbonate rocks, diabase, dolomite, eruptive rocks, feldspar, ground glass, glasses, mica, gneiss, greywacke, silica earths, diatomite, silicic acid, chalk, lava rocks, magnesite, metal oxide rocks, meteorite rocks, montmorillonite, pyrite, quartz, quartz sand, slate, sedimentary rocks, silicate rocks, sulfate rocks, clays, clay rocks, trass, tuffs, volcanic ashes, volcanic rocks, and mixtures thereof.

According to a preferred embodiment of the invention, the inorganic solid particles are embedded in the matrix material formed by the polymer and/or the inorganic solid particles are bound to the matrix material formed by the polymer.

According to a preferred embodiment of the invention, the inorganic filler particles are present in amounts of ≧10 to ≦95 wt.-%, particularly ≧30 to ≦90 wt.-%, preferentially ≧50 to ≦85 wt.-%, with reference to the biocide improvement system.

According to a preferred embodiment of the invention, the inorganic filler particles have particle sizes (absolute) of ≦2,000 μm, particularly ≦1,000 μm, preferentially ≦500 μm, particularly preferably ≦250 μm, where at least ≧95%, preferably at least ≧99% of the inorganic filler particles lie within the aforementioned value range.

Alternatively or supplementally, the inorganic filler particles preferably have particle sizes (absolute) in the range of ≧1 nm to 2,000 μm, particularly ≧10 nm to ≦1,000 μm, preferentially ≧20 nm to ≦500 μm, particularly preferably ≧50 to ≦250 μm, where at least 90%, preferentially at least 95%, preferably at least 99% of the inorganic filler particles lie within the aforementioned value range.

According to a preferred embodiment of the invention, the biocide improvement system has a residual monomer content of less than 1 wt.-%, particularly less than 0.5 wt.-%, preferentially less than 0.3 wt.-%, particularly preferably less than 0.1 wt.-%.

According to a preferred embodiment of the invention, the biocide improvement system is configured to be pourable, particularly flowable; this has proven to be particularly advantageous for many applications, because the processability is increased in this way.

Alternatively or supplementally, the biocide improvement system preferably has a bulk density in the range of ≧200 to ≦1500 g/l, particularly ≧500 to ≦1000 g/l, preferentially ≧550 to ≦900 g/l, particularly preferably ≧600 to ≦800 g/l.

Alternatively or supplementally, the biocide improvement system preferably has a pH, when water is added, in the range of ≧4 to ≦8, particularly ≧5 to ≦7.

Alternatively or supplementally, the biocide improvement system preferably has a conductivity of less than 5,000 μS/cm, particularly less than 3000 μS/cm, preferentially less than 2000 μS/cm.

Alternatively or supplementally, the biocide improvement system is preferably processed to produce molded bodies, particularly pellets, grains, beads, granulates, disks, lamellae, or the like.

Particularly preferably, the biocide improvement system is configured in particle form, and particularly has a grain size (absolute) in the range of 0.01 to 20 mm, particularly 0.1 to 10 mm, preferentially 0.5 to 8 mm, where at least 80%, preferentially at least 85%, preferably at least 90% of the particles of the biocide improvement system lie within the aforementioned value range.

Particularly preferably, the biocide improvement system possesses a time-dependent swelling behavior in distilled water, particularly where the biocide improvement system possesses a water absorption (in distilled water, in each instance) within one hour of at least 5 times, particularly at least 10 times, preferentially at least 15 times, particularly preferably at least 20 times its own weight and/or that the biocide improvement system possesses a reversible water absorption and/or water storage capacity.

Particularly preferably, the biocide improvement system has a weight-related water absorption capacity overall, with reference to the weight of the biocide improvement system, of at least 500%, particularly at least 1,000%, preferentially at least 1,500%, particularly preferably at least 2,000%.

The present invention furthermore relates to a production method for the production of the biocide system according to the invention, comprising the step of polymerization of the inorganic polymer from suitable precursor substances in the presence of the carbohydrate-based structural units, the inorganic solid particles, as well as the biocide.

Surprisingly, it has been shown that by means of this simple production method, the biocide is bound to the matrix material in such a reversible manner (whether physically or chemically, as described above) that the advantages according to the invention can be achieved.

The aforementioned components as well as those claimed and those described in the exemplary embodiments, to be used according to the invention, are not subject to any particular exceptional conditions in terms of their size, shape configuration, material selection, and technical conception, so that the selection criteria known in the field of use can apply without restriction.

Further details, characteristics, and advantages of the invention are evident from the dependent claims and from the following description of the related example, which should be understood purely as an illustration and not as restrictive.

### 1. Production of a Biocide Improvement System According to the Invention

A solution containing 4.4 g of urea, 162 g of tap water having a degree of hardness of 20 dH, and 100 g acrylic acid are added to an approximately 1 liter conical plastic vessel. Thirty-four grams of a potassium hydroxide solution (50.0 wt. %), 15.0 g of potassium water glass, and 10.0 g molasses are added to the mixture. The mixture is cooled to below 10 degrees C., and a mineral substance mixture containing 124.0 g of fine quartz sand, 124.0 g of Eifelgold and 62.0 g of bentonite are stirred into this solution. Polymerization is initiated with initially strong stirring and the addition of 0.06 g potassium disulfite and 1.62 g sodium peroxide disulfate, each in the form of a saturated solution. Within a few minutes, the mass fills the entire plastic vessel and forms a mushroom above the vessel.

After cooling, the polymer block is removed and a small slice cut from it, having a weight of 1.2 g, is placed in tap water at 20 dH. The weight increase over time can range from about 5 to about 20 times the sample's weight.

The remainder is dried to a residual moisture of approx. 30% and subsequently ground (grain size: 2 to 6 mm). The bulk density lies at 650 to 680 g/l, the pH (10% water) lies at 5 to 7, and the conductivity lies below 1,000 [mu]S/cm. The residual monomer content lies below 0.1 wt.-%. Half of the batch resulting in this manner is processed to produce molded bodies (pellets at approx. 10 mm).

Thiamethoxam (TMX) was used as a biocide; it has the following structure:

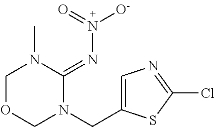

The IUPAC name is 3-[(2-chloro-1,3-thiazol-5-yl)methyl]-5-methyl-N-nitro-1,3,5-oxadioazinan-4-imine. It is used, among other things, as an insecticide against aphids.

2. Experiments Concerning Efficacy/Penetration Depth into Soils

The penetration depth of TMX with and without the improvement system was investigated using the test setup described below.

In this connection, a cylinder (diameter approx. 5.5 cm) was filled to approx. 21 cm with liquid-saturated soil. The soil was divided into three zones of 7 cm each, which were called Zone I (uppermost soil layer), Zone II (middle soil layer), and Zone III (bottommost soil layer).

Subsequently, the cylinder was filled up with 6 cm soil that had been mixed once with 0.5 mg/ai TMX (comparison test), and once with 6 cm soil with 0.5 mg/ai TMX and 2 kg/m² improvement system (invention example).

The cylinder was now watered (watering rate 255 l/m²) until the water had reached the bottom, i.e. complete watering had been achieved.

Subsequently, the soil of Zone I (uppermost zone) to Zone III (bottommost zone) was removed, and chickpeas that had been infected with aphids (Aphis craccivora) were planted in the soil. Now the mortality rate of the aphids was measured, as listed in Table I (average values on the basis of multiple experiments).

TABLE I

| | Zone I | Zone II | Zone III |
|---|---|---|---|
| Example according to | 85% | 5% | 5% |
| the invention | | | |
| Comparison example | 93% | 70% | 5% |

It can be clearly seen from the table that the improvement system reduces penetration of the insecticide into deeper soil layers (=Zone II), i.e. the insecticide essentially remains in the higher soil layers, where it is also most effective.

The individual combinations of the components and the characteristics of the embodiments already mentioned are given as examples; replacement and substitution of these teachings with other teachings that are contained in this document with the cited documents are also explicitly considered. A person skilled in the art recognizes that variations, modifications, and other embodiments that are described here can also occur, without deviating from the idea of the invention and the scope of the invention. Accordingly, the above description should be viewed as an example and not as restrictive. The word used in the claims comprises and does not exclude other components or steps. The indefinite article “a/an” does not exclude the meaning of a plural. The mere fact that specific dimensions are recited in claims that differ from one another does not mean that a combination of these dimensions cannot be used to advantage. The scope of the invention is defined in the following claims and the related equivalents.

1. A biocide improvement system for use in soils, comprising
a water-swelling matrix material on the basis of at least one organic polymer, which comprises carbohydrate-based structural units, particularly carbohydrate-based functional groups,
wherein inorganic solid particles are added to the matrix material,
and comprising at least one biocide reversibly bound to the matrix material and/or to the inorganic solid particles.

2. The biocide improvement system according to claim 1, wherein the biocide is a fungicide, herbicide or insecticide.

3. The biocide improvement system according to claim 1, wherein the concentration of biocide comprises from 0.0001% to 10% by weight.

4. The biocide improvement system according to claim 1, wherein the biocide is selected from the group containing aliphatic nitrogen compounds, antibiotic fungicides, macrocyclic lactones, amides, anilides, particularly acylamino acids, inorganic herbicides, chloroacetanilides, phenoxynicotinanalides, sulfonanilide, anilino-pyrimidines, aryloxyphenoxypropionates, aryl-phenyl-ketones, azoles, benzofuranylalkylsulfonate, benzothiazole, bipyridyls, carbamates, carbanilates, growth regulators, chitin synthesis inhibitors, chlorinated hydrocarbons, cyclohexanediones, cyclohexene oximes, diazoles, dichlorophenyl dicarboximides, dinitroanilines, dinitrophenol, diphenyl ethers, dithiolanes, halogenated aliphates, urea derivatives, hydrazides, imidazolinones, Juvenile Hormone, synthetic Juvenile Hormones, coumarins, morpholines, neonicotinoids, nitriles, nitrophenyl ethers, organophosphates, organothiophosphates, oxadiazoles, oxazolidinone derivatives, phenoxy herbicides, phenyl pyrazolines, pheromones, phosphoric acid esters, phthalimides, Precocene, pyrazoles, pyrethroids, pyridazines, pyridazinones, pyridines, pyromidines, pyrroles, quaternary ammonium salts, quinolines, quinoxalines, quinones, strobilurins, sulfite esters, sulfonyl ureas, synthetic auxins, tetrazines, tetronic acid, thiadiazines, thiophene, thiazolidines, triazines, triazinones, triazoles, triazolones, or mixtures thereof.

5. The biocide improvement system according to claim 1, wherein the biocide is selected from the group containing (E)7-(Z)9-dodecadienyl acetate, abamectin, aclonifen, acrolein, alpha-cypermethrin, aluminum phosphide, amidosulfuron, azoxystrobin, bendiocarb, bentazon, benzofuranyl methylcarbamate, beta-cyfluthrin, Bitrex, boroxide, boric acid, boscalid, bromadiolone, bromoxynil, bromoxynil octanoate, captan, carbaryl, carfentrazone-ethyl, chalcogran, chloralose, chloridazon, chloromequat chloride, chlorothalonil, chlorophacinone, clodinafop propargyl, clomazone, cloquintocet-mexyl, clothianidin, codlemone, coumatetralyl, cycloxydim, cyproconazole, cyprodinil, dazomet, diquat, diquat bromide, deltamethrin, desmedipham, dicamba, dichofluanid, dichloroprop-P, dichlorvos, difenacoum, difenoconazole, difethialone, diflufenican, dimethachlor, dimethenamid-P, dimethoate, dimethomorph, dimethyl carbamate dimoxystrobin, disodium octaborate tetrahydrate, disodium tetraborate, dithianon, epoxiconazole, esfenvalerate, etofenprox ethephon, ethofumesate, ethylenediamine hydroxyphenyl acetic acid, etofenprox, fenhexamid, fenoxycarb, fenpropidin, fenpropimorph, flocoumafen, florasulam, fluazifop-P-butyl, fluazinam, fludioxonil, flufenacet, fluopicolide, fluoxastrobin, fluquinconazole, fluoroxypyr, flurtamone, folpet, foramsulfuron, fosetyl-A1, fosthiazate, fuberidazole, glufosinate ammonium, glyphosate, hexythiazox, imazalil, imidacloprid, indoxacarb, 3-iodo-2-propynylbutylcarbamate, iodosulfuron methyl sodium, ioxynil, iprodione, iprovalicarb, isoproturon, isoxadifen ethyl, kresoxim methyl, lambda cyhalothrin, magnesium phosphide, mancozeb, mandipropamid, mefenpyr-diethyl, mepiquat chloride, mesosulfuron methyl, mesotrione, metafluizone, metalaxyl-M, metaldehyde, metazachlor, metconazole, methiocarb, methofluthrin, methoxyfenozide, metiram, metosulam, metrafenone, metribuzin, napropamide, N,N-diethyl-m-toluamide, penconazole, pencycuron, penoxsulam, pethoxamid, phenmedipham, picolinafen, pinoxaden, piperonyl butoxide, pirimicarb, pirimiphos methyl, prochloraz, prochloraz copper chloride, prohexadione calcium, propamocarb HCl, propiconazole, propoxycarbazone, propylene glycol, proquinazid, prosulfocarb, prosulfuron, prothioconazole, pymetrozine, pyraclostrobin, pyrethrin, pyrimethanil, quinmerac, quizalofop-P, glyphosate, S-cis-verbenol, S-ipsdienol, S-metolachlor, spinosad, spirodiclofen, spiroxamine, sulfuryl fluoride, sulcotrione, tebuconazole, tebufenpyrad, tefluthrin, tembotrione, tepraloxydim, terbuthylazine, thiabendazole, thiacloprid, thiamethoxam, thiophanate methyl, tolylfluanid, topramezone, triasulfuron, trifloxystrobin, triflumuron, trinexapac-ethyl, triticonazole, tritosulfuron, warfarin, warfarin sodium, A-9 dodecenyl acetate, zinc phosphide, or mixtures of these substances.

6. The biocide improvement system according to claim 1, wherein the organic polymer is configured to be cross-linked.

7. The biocide improvement system according to claim 6, wherein cross-linking is brought about by means of difunctional cross-linking agents.

8. The biocide improvement system according to claim 1, wherein the organic polymer contains or comprises a polymer that contains a carboxyl group.

9. The biocide improvement system according to claim 1, wherein the organic polymer possesses a structure that is sponge-like, porous, and/or has cavities.

10. The biocide improvement system according to claim 1, wherein the inorganic component is selected from the group of basalt, bentonite, pumice, calcite, carbonate rocks, diabase, dolomite, eruptive rocks, feldspar, ground glass, glasses, mica, gneiss, greywacke, silica earths, diatomite, silicic acid, chalk, lava rocks, magnesite, metal oxide rocks, meteorite rocks, montmorillonite, pyrite, quartz, quartz sand, slate, sedimentary rocks, silicate rocks, sulfate rocks, clays, clay rocks, trass, tuffs, volcanic ashes, volcanic rocks, and mixtures thereof.

11. The biocide improvement system according to claim 6, wherein the organic polymer is configured to be structurally cross-linked.

12. The biocide improvement system according to claim 7, wherein cross-linking is brought about by means of diol.
